(19) United States
(12) Patent Application Publication
(10) Pub. No.: US 2002/0059029 A1
(43) Pub. Date: May 16, 2002

(54) Method for the diagnosis of thought states by analysis of interword silences

(76) Inventors: Doran Todder, D.N. Hanegev (IL); Sofia Schreiber-avissar, Omer (IL)

Correspondence Address: MERCHANT & GOULD PC, P.O. BOX 2903, MINNEAPOLIS, MN, 55402-0903, US

(21) Appl. No.: 09/902,296

(22) Filed: Jul. 10, 2001

Related U.S. Application Data
(63) Continuation of application No. PCT/IL00/00019, filed on Jan. 10, 2000.

(30) Foreign Application Priority Data
Jan. 11, 1999 (IL) 128000

(51) Int. Cl. G06F 19/00; G01N 33/48; G01N 33/50; G10L 15/00; G10L 15/04

(52) U.S. Cl. 702/19; 704/251

(57) ABSTRACT

Method and apparatus for the analysis of thought states in a subject. The voice of the subject is recorded during his speech into a memory. The recorded voice is digitized and the gain of the electrical signals representing the subject's voice is controlled. The digitized voice is transformed into a digital data representation and the interword time intervals (ITIs) are measured from the digital data. Parameters representative of the subject's ITI behavior are extracted from the measures ITI data and the subject is then characterized by processing and analyzing his ITI data using the extracted parameters. The characterization of the subject is carried out by calculating the correlation dimension based on his ITI data.

### FIELD OF THE INVENTION

The present invention relates to the field of the diagnosis of mental processes. More particularly, the invention relates to a method for the diagnosis of psychoses, of cognitive development stages, and of mental processes in general (collectively referred to as “thought states”), by the analysis of measured data from interword time intervals of the patient's speech.

### BACKGROUND OF THE INVENTION

The psychoses form a group of psychiatric disorders characterized by gross distortion of mental capacity, affective response, and capacity to recognize external reality, even in the face of contrary evidence. This diagnostic group includes several major psychiatric illnesses such as schizophrenia and bipolar disorder. While some of the psychoses are of mainly organic origin, many others are less obviously so. Study of the latter group of disorders open, therefore, has to be made in relation to functional aspects such as the type of thought processes used. In this respect, it is interesting to note that normal children up to the age of approximately eight years, use thought processes very similar to those seen in psychotic disorders (e.g. “magical” thinking, rather than logical thinking). The transition from this psychotic-like thinking to the normal adult pattern thus represents a major developmental milestone.

Several attempts have been made to identify quantifiable factors which may serve as the basis for more objective tests for the diagnosis of psychoses. A number of such studies have been directed toward the use of speech analysis for the diagnosis of schizophrenia and other psychoses. These have invariably focused on the analysis of words as vocal signals (Stassen et al. Psychopathology 24: 88-105 (1991)). However, the results of such analyses are strongly related to content (i.e. the spoken message) and may be language- and culture-dependent.

Other disorders connected with mental processes are also difficult to quantify. These include, e.g., cognitive development stages and other anomalies or behavioral disfunctions connected with mental processes or cerebral activity,

It has now been further found, and this is an object of the present invention, that the interword time intervals contain critical information that makes it possible to carry out diagnostic measurements of thought states, that are unrelated to speech content. Such analyses are much purer measurements of the form of speech than prior art methods, and as such, provide a much more robust basis for the development of reliable diagnostic tests. It should be noted that it is extremely surprising that interword time intervals, entirely unrelated to thought processes may provide an indication of abnormal thought states. This surprising discovery permits to obtain the results achieved by the invention.

It has now been further surprisingly found, and this is another object of the invention, that it is possible to use fractal analysis of the dynamic patterns present in a string of interword silences as a diagnostic tool for the psychoses.

It is an object of the invention to provide a method for the diagnosis of thought states, which is based on silences between words in potential patients speech.

It is another object of the invention to provide a method for the diagnosis of psychoses, which is based on silences between words in potential patients speech.

It is still another object of the invention to provide a method for determining the cognitive development stage in healthy children.

It is a further object of the invention to provide a method for the analysis of speech interword pauses.

It is yet another object Al the invention to provide apparatus for the analysis of speech interword pauses.

Other objects of the invention will become apparent as the description proceeds.

### SUMMARY OF THE INVENTION

The present invention is directed to a method for the diagnosis of thought states. In the context of the present invention, by “thought states” it is meant to indicate any condition that affects the interword time intervals of a person's speech, be it a physiological or non-physiological problem, whether permanent or temporary. Such thought states include, inter alia, psychoses and cognitive development stages, which are diagnosed by the analysis of measured data from interword time intervals of the patient's speech. The voice of the subject is recorded, stored in a memory and digitized. The gain of the electrical signals representing the subject's voice is controlled, and the digitized voice is converted to a digital data representation. The interword time intervals (ITIs) are measured from the digital data and the parameters which are representative of the subject's ITI behavior are extracted from the measured ITI data. The subject is then characterized by processing and analyzing his ITI data using the extracted parameters.

Preferably, the subject is characterized by calculating the correlation dimension based on his ITI data. The embedding dimension is determined and the values of the original vector are normalized to a unity time interval. A set of state vectors is generated from the original vector. The dimension of each vector is equal to the embedding dimension. A state vector represents a part of the original ITI data that has been cut according to the embedding method.

Preferably, a threshold distance between a pair of state vectors is determined, below which the pair of state vectors are correlated. The correlation integral for said set of state vectors is calculated as a function of a predetermined range of threshold distances. The calculated values of said correlation integral are plotted as a function of the predetermined range of threshold distances using a logarithmic-logarithmic scale, and a linear region in the logarithmic-logarithmic plot is sought The mean value of all local slopes within the identified linear region is calculated. This process is repeated for a plurality of different embedding dimensions which are higher than the determined embedding dimension. The correlation dimension function is obtained by plotting all mean values of all local slopes, calculated for each embedding dimension, as a function of the embedding dimension. The random or deterministic attributes of the original vector may be characterized by identifying convergence or divergence of the correlation dimension function with a growing value of the embedding dimension.

Preferably, the patient is characterized by a symbolic dynamics analysis of his ITI data. The range of the ITI data is divided into a set of data intervals, and a unique symbol is assigned to each data interval. The set data values from the original vector is transformed into a corresponding set of symbols by the ascription of each data value, contained in the interval, to a corresponding interval from the set of data intervals. A group of symbols of fixed size is defined from the set of symbols, and the frequency of the defined group is identified by calculating the Shannon entropy for the group of symbols. The Shannon Entropy is used to find differences between the group of normal subjects and the group of psychotic patients. An up-going series is generated from the calculated Shannon entropy values (as hereinafter defined), and finally, the random or deterministic attributes of the original vector is determined by applying the Mann-Whitney test on the generated up-going series and finding differences between the group of normal subjects and the group of psychotic patients. Mann-Whitney test is a type of non-parametric statistical test, indicating a statistical difference between two groups of numbers. The result of the test is a pair of values <P,U>, wherein P represents the probability that the two groups are different and U provides an indication about the size of the examined groups. Mann-Whitney test is disclosed, for example, in “Glantz, S. A. Biostatistics”, 3^{rd} ed. New York, 1992.

Preferably, the subject is fiber characterized by finding and counting points of Unstable Periodic Orbits (UPOs), based on his ITI data. A three dimensional phase space, containing a plurality of points that are related to the values of the original vector is constructed. All points of UPOs in the original vector are identified and counted by seeking all sets of six consecutive points in the phase state, for which the corresponding distances of the first three points from the first set to the main diagonal defines a down-going series, and the corresponding distances of the last three points from the first set to the main diagonal defines an up-going series. A surrogate vector is generated from the original vector by randomly scrambling the order of its data values and all points of UPOs in the surrogate vector are identified and counted. This process is repeated several times and finally the mean value of all counts of points of UPOs is calculated over all generated surrogate vectors. The number of points of UPOs, identified in said original vector is used to characterize the random or deterministic attributes of the original vector.

Preferably, the subject may be characterized by performing bi-spectral analysis of his ITI data. The inter-modulation products of the sampled ITI data is computed, as well as the tripe product for each pair of Fourier frequency components of the inter-modulation products. The computed triple products of all pairs of Fourier frequency components are summed and the bi-spectrum is obtained from the magnitude of the sum. The Real-Triple Product of all inter-modulation products are computed and the bi-spectrum is normalized to the Real-Triple Product. A two-dimensional contour graph of the normalized bi-spectrum is generated and plotted as a function of Fourier frequencies. The number of closed contours provides an indication about psychoses.

The invention is also directed to an apparatus for the diagnosis of thought states by the analysis of Interword Time Intervals (ITIs). The apparatus comprises:

a) a microphone for converting the patient's voice to a series of electric signals;

b) an Automatic Gain Control (AGC) circuitry for controlling the level of the electric signals representing the speech data;

c) a Coder-Decoder (CODEC) for digitizing the speech data;

d) a converter, for transforming the digitized speech data into digital representation;

e) a digital signal processor (DSP) for measuring the speech ITIs, analyzing the data and extracting the required parameters for further ITI analysis;

f) a memory associated with the digital signal processor, storing the digital speech data information together with the processed data;

g) a second memory, storing the parameters extracted according to step e) above;

h) analysis and computation unit for patient characterization based on the ITI data of step f) above and the stored parameters of step g) above;

i) user interface means for user interaction with analysis and computation unit;

j) communication means communicating between the digital processor with its associated memory, the user interface and the analysis and computation unit;

k) a controller associated with the second memory of step g) above, for controlling the operations of the AGC circuitry, the DSP and its associated memory and the communication means of step j) above;

l) display and/or printing means for displaying analysis results and/or the patient characterization parameters; and

m) optional voice playback means for representing the analysis and computation unit results.

### BRIEF DESCRIPTION OF THE DRAWINGS

The present invention will be more clearly understood from the following detailed description of preferred embodiments thereof, with reference to the appended drawings, wherein:

FIG. 1 is an example of a digital recording of the sentence: “The sun is shining” showing both the spoken words and the Interword Time Intervals (ITIs);

FIGS. 2A and 2B are plots of ITIs recorded from a healthy subject, and from a psychotic patient, respectively;

FIG. 3 is a logarithmic plot of the in vector's correlation integral and the mean slope of that integral for an embedding dimension value of two, according to a preferred embodiment of the invention;

FIG. 4 is a logarithmic plot of the correlation integral for embedding dimension values ranging from 2-14, according to a preferred embodiment of the invention;

FIG. 5 is a plot of the mean slope values of the plot of FIG. 4, according to a preferred embodiment of the invention;

FIG. 6 is a representative plot of the correlation integral (C(r,n)) as a function of n, the embedding dimension for the ITIs of a normal subject (closed circles). A surrogate record (examined in Example 1) of the same ITIs (open circles) resulted in a non-saturable curve, indicating a random time series;

FIG. 7 is a representative plot of the correlation integral (C(r,n)) as a function of n, the embedding dimension for the ITIs of a psychotic patient (closed circles). The open circles represent a surrogate record of the same ITIs;

FIG. 8 is a plot of the Shannon entropy calculated from the distribution of selective words in the symbolic dynamics of interword time intervals of the speech of normal individuals and psychiatric patients, resulted from Mann-Whitney test with U=31 and P=0.0012;

FIG. 9 is a plot of correlation dimension and symbolic dynamics entropy as a function of age in a group of children aged 6 to 14 years;

FIG. 10 is a block diagram of an apparatus for the diagnosis of psychotic patients by the analysis of Interword Time Intervals (ITIs), according to a preferred embodiment of the invention;

FIGS. 11A and 11B are three-dimensional contour plots obtained from bi-spectral analysis of the speech of normal individuals and psychiatric individuals respectively;

FIG. 12 is a graph of the distribution of closed contour circle characteristics derived from bi-spectral analysis, between 15 psychiatric patients and 15 normal individuals;

FIG. 13 is a plot of circle areas obtained from contour plots of data derived from bi-spectral analyses of normal and psychiatric patients, resulted from Mann-Whitney test with U=58.5 and P=0.042;

FIG. 14 is a graph showing the correlation between three analytical methods (bi-spectral, correlation dimension and symbolic dynamics) applied to normal individuals and psychiatric patients;

FIG. 15 shows in greater detail the correlation between bi-spectral analysis and symbolic dynamics measurements (upper right panel of FIG. 14); and

FIG. 16 is a regression plot giving the relationship of symbolic dynamics analysis with child age.

### DETAILED DESCRIPTION OF PREFERRED EMBODIMENTS

For the purposes of clarity, and as an aid in the understanding of the invention, the following terms and abbreviations are defined below:

Unstable periodic orbits—graphical representation of instability of a dynamic system by forming periodic orbits in its state plane. Unstable periodic orbits are disclosed, for example, in “Chaos in dynamical systems, E. Ott, Cambridge University, 1989”

Chaotic attractor—a graphical method for indicating acts and characteristics of a chaotic system (a system which acts in an unpredictable manner which is very sensitive to initial conditions) in a state-space. Chaotic attractor is disclosed, for example, in “Chaos in dynamical systems, E. Ott, Cambridge University, 1989”, in “Nonlinear dynamics and chaos, S. H. Stogattz, Addison-Wesley, 1994”, and in “Chaos and psychology: Deterministic chaos in excitable cell assemblies, T. Elbert et al., The American physiological society, 74, 1, 1994”.

Shannon entropy—an information function which indicates the information required for a system to infer about the future state of the system. Mathematically, if pᵢ is the probability of the i-th state, the Shannon entropy is given by

$$S = - {\sum }_{i} {p}_{i} \cdot ln ( {p}_{i} ) .$$

The Shannon entropy is discussed, for example, in “Exploring Complexity, G. Nicolis and I. Prigogine, W. H. Freeman and Company/New York, 1989”.

According to a preferred embodiment of the invention, the diagnosis of the thought state, e.g., a psychosis, in a subject is made by performing the fractal analysis of the ITIs taken from a recording of the subject's speech. The value of the correlation dimension at saturation (dₛ) is compared with those derived from corresponding analyses performed on two reference groups:

(i) normal subjects, and

(ii) (in this specific example) psychotic patients.

According to another preferred embodiment of the invention, ITI analysis is performed by the calculation of the Shannon entropy from symbolic dynamics analysis. The value of the Shannon entropy obtained for the test subject is compared with those calculated for two reference groups:

(i) normal subjects, and

(ii) (in this specific example) psychotic patients.

In yet another preferred embodiment of the invention, the methods of correlation dynamic analysis, and symbolic dynamics analysis, are applied to ITIs obtained from speech recordings of children. Assessment of the stage of cognitive development reached by a child is made by comparison of the results of these analyses with those obtained from a reference group of normal children

According to still another preferred embodiment of the invention, the diagnosis of the thought state (e.g., a psychosis) in a subject is made by obtaining UPOs from analysis of the ITI data vector, taken from a recording of the subject's speech and analysis of surrogate vectors, which are generated by randomly scrambling the data of the ITI vector. The number of the UPOs obtained from the ITI vector is compared with those derived from the surrogate vectors. The comparison ret provides an indication of whether the ITI is deterministic or random.

According to a preferred embodiment of the invention, the ITI vector is analyzed using bi-spectral analysis, which is a signal processing technique that quantifies non-linearities and deviation from normality (i.e., deviation from a Gaussian distribution of the signal amplitude). Bi-spectral analysis is disclosed, for example, in “Introduction to bi-spectral analysis, Sigal and Chamoun, Journal of clinical monitoring, 10, 6, 396, 1994”. Since the ITI vector represents the output of a non-linear system, it consists of a plurality of Inter-Modulation Products (IMP) of input signals (i.e., Fourier components of the input signal), which are of different frequencies and phases. Since the phase angles of the IMPs are dependent on the phase angles of these Fourier components, they are “phase-coupled”. The bi-spectal analysis is used to characterize the degree of phase coupling. The bi-spectrum is computed by computing the triple product X(f₁)X(f₂)X^{*}(f₁+f₂) for each pair (f₁, f₂) of Fourier frequency components, (X(f) represents the Fourier transform of the sampled input signal), summing all products and taking the magnitude of the sum. The resulting bi-spectrum, which is sensitive to the phase-coupling, is normalized to the Real Triple Product (i.e., the triple product with perfect phase-coupling). A high degree of phase-coupling results in higher bi-spectal (and higher normalized bi-spectral) product.

Important information may be extracted from phase coupling of the ITI vector. The non-linear processes that generate the ITI vector comprise several occurrences of phase-coupling. Obtaining the number of occurrences provides an indication whether the ITI vector is deterministic or random.

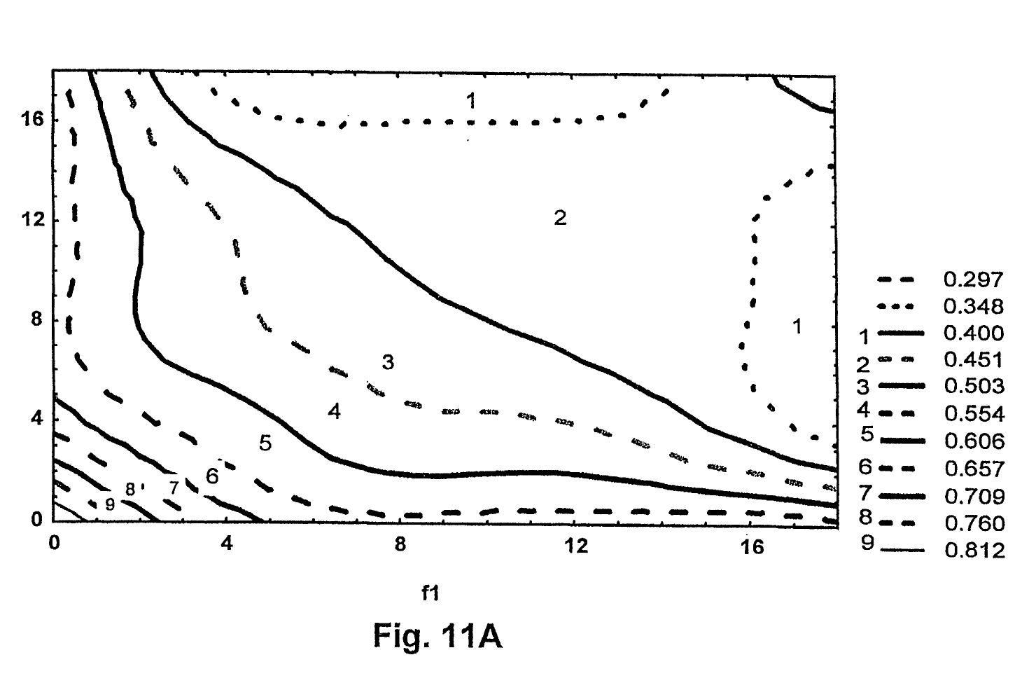

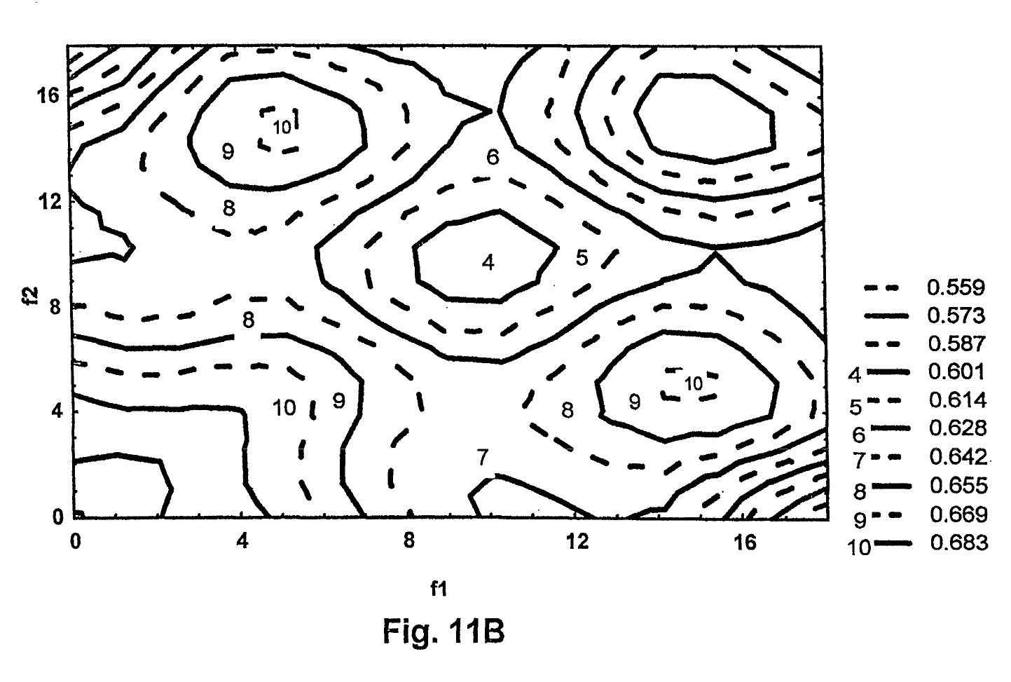

FIG. 11A and 11B are three-dimensional contour graphs, showing the normalized bi-spectral product of normal and mentally ill subject, respectively, as a function of f₁ and f₂. The two contours are totally different in structure. The contours that represent the mentally ill subject (shown in FIG. 11B) form several closed circles, while no closed circles are formed by the contours that represent normal subjects, as can be seen from FIG. 11A.

According to a preferred embodiment of the invention, the values which are obtained from bi-spectral analysis of the ITI vector, are compared with the values which are obtained from bi-spectral analysis of a surrogate (file) ITI vector. Since the value of the phase-coupling of a surrogate ITI vector is much lower than the original ITI vector because of random characteristics), the comparison result provides an indication about the probability that the original ITI vector has a random characteristic.

The following examples are illustrative of this invention. They are not intended to be limiting upon the scope thereof.

### EXAMPLE 1

### Low-Dimensional Dynamics in Interword Time Intervals

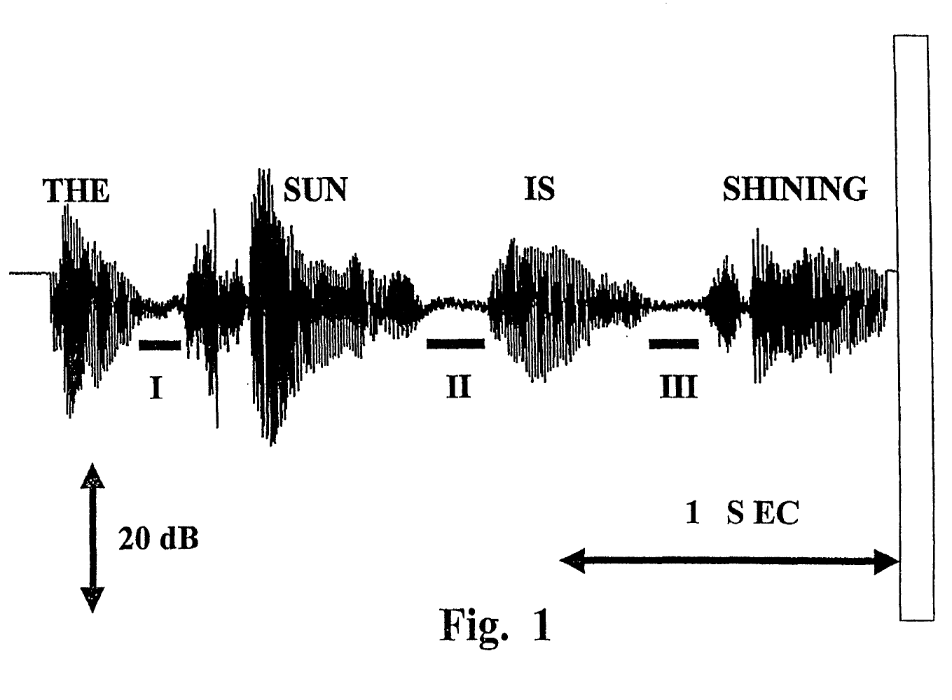

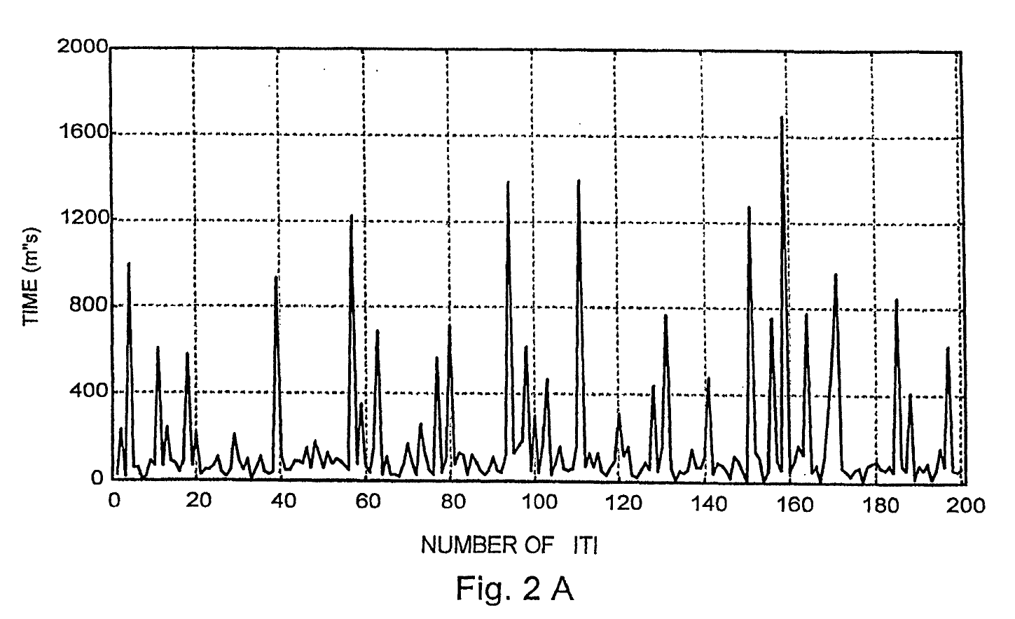

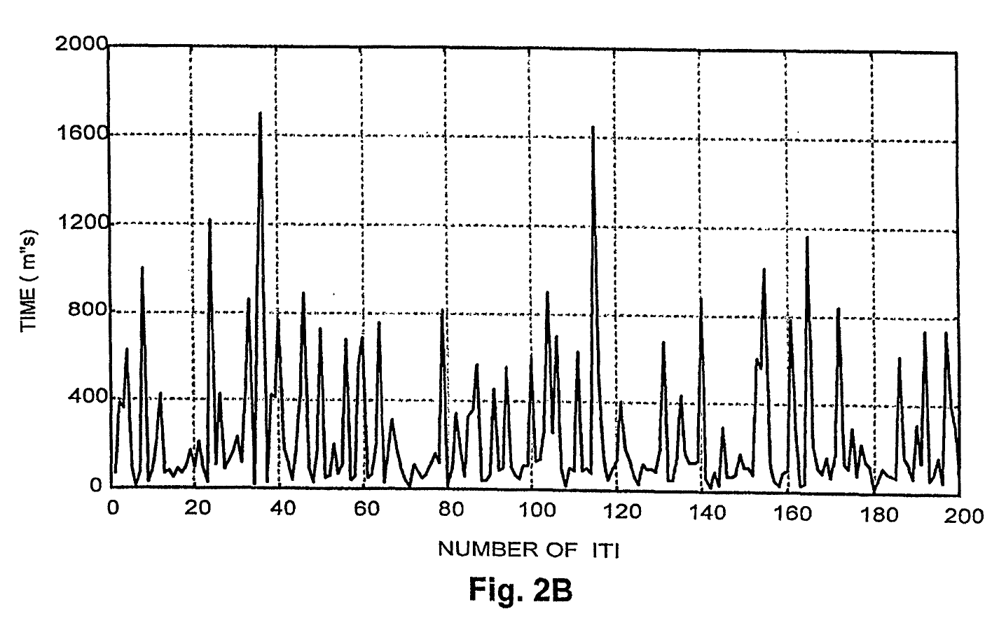

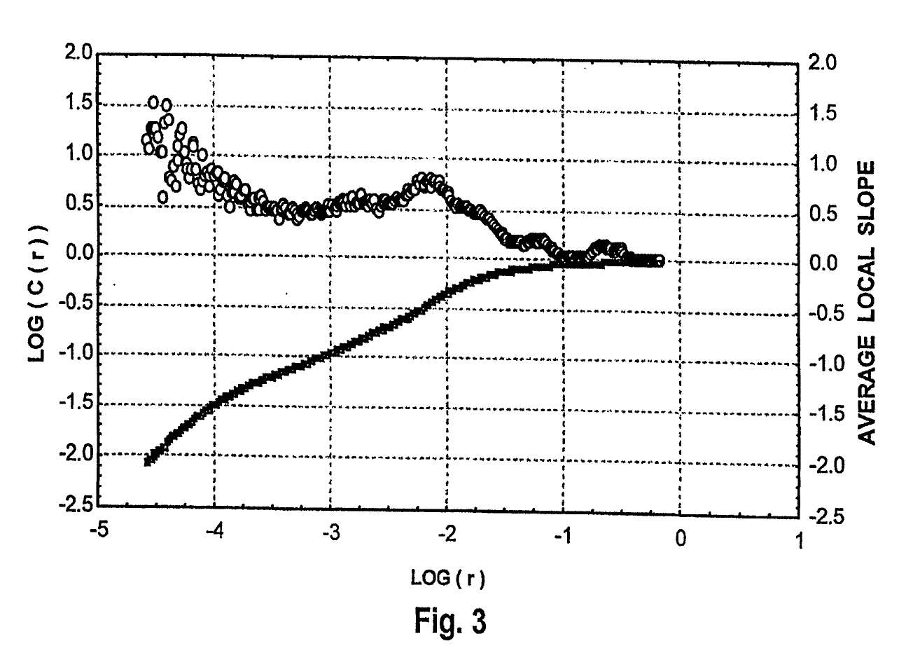

A group of 15 normal subjects and 15 untreated psychotic patients were recorded while freely speaking for 10 to 15 minutes. Their recorded speech was analyzed for the series of interword time intervals (ITI). FIG. 1 is an example of a digital recording of the sentence: “The sun is shining” displaying both the spoken words and the ITIs. Representative plots of ITI recorded from (A) a healthy subject, and (B) a psychotic patient, are shown in FIG. 2. These recordings were scanned for Usable Periodic Orbits (UPOs), and the number of UPOs within 100 successive ITIs (N) was counted.

Normally, analysis based on UPOs is carried out by obtaining unique events in a measured vector, for inferring about random or deterministic character of the measured vector. A chaotic system may comprise points of UPO (considered as unique events), i.e., points in which the chaotic system can be strongly affected. Obtaining such points of UPOs in an experimental system is an indication to a deterministic character of the system. According to a preferred embodiment of the invention, points of UPOs are calculated according to the following steps. At the first step, a three-dimensional (3-D) phase space is constructed by the Embedding Technique, resulting in a 3×n dimensional matrix, wherein each row in the matrix represents a point in the phase space. At the next step, three consecutive points are sought in the phase space, such that their corresponding distance to the main diagonal (the diagonal, stating from the origin (x=y=z=0), for which every point has an identical value from each axis, i.e., x=y=z), is down-going. At the next step, the corresponding distance of the next three consecutive points to the main diagonal is checked. If the latter corresponding distance is up-going, and the slopes of the line connecting between the two obtaind peaks and the line connecting between the two obtained descents are essentially orthogonal, the combination of these six points generates a “saddle” in a 2-D topographic domain, which represents a point of UPO. At the next step, all points of UPOs in the measured vector are identified and counted. At the next step the vector values are randomly scrambled and placed in a different order. Hence, a new vector with the same values of distribution, mean value and standard deviation as the original vector but with a different order, is generated. At the next step all the points of UPOs of the new vector are calculated, counted and compared to the number of points of UPOs in the original vector. This (random scrambling) process is repeated for a predetermined number of times, and finally, the mean value of the number of points of UPOs of all scrambled vectors is compared to the number of points of UPOs in the original vector. A substantial difference provides an indication tat the original vector is deterministic.

In the experimental example of the invention, this process was repeated in 100 surrogate records which were constructed using 100 different realizations of the randomization process. Ns is the average number of UPOs within 100 interword intervals in the surrogate records. A measure of significance is defined by the difference between the original and the surrogate value of the statistic (N and Ns respectively) divided by the standard deviation of the surrogate values (σ):

K=(|N−Ns|)/σ (1)

Assuming Gaussian statistics, the p value is given by:

p=erfc(K/2) (2)

These results show that 60-70% of ITIs, both in normal and in psychotic subjects, showed statistically significant (p<0.05) more encounters with UPOs in the original data than their surrogates. Tables 1 and 2A comprises an example of UPO calculation results.

TABLE 1

| Psychotic Patients | | | | Normal Subjects | | | |
|---|---|---|---|---|---|---|---|
| | | Mean | No. of | | | Mean | No. of |
| p | σ | Value | UPOs | p | σ | Value | UPOs |
| .012 | .91 | .68 | 3 | .03 | .67 | .54 | 2 |
| 10⁻⁴ | .87 | .67 | 4 | .003 | .79 | .76 | 3 |
| .046 | .73 | .54 | 2 | .004 | .8 | .72 | 2 |
| 8 · 10⁻⁴ | .74 | .51 | 3 | .044 | .71 | .56 | 2 |
| 3 · 10⁻⁶ | .63 | .38 | 3 | 4 · 10⁻⁴ | .71 | .52 | 3 |
| .05 | .74 | .55 | 2 | .02 | .65 | .49 | 2 |
| .0085 | .86 | .73 | 3 | .05 | .77 | .54 | 2 |
| .04 | .71 | .53 | 2 | .009 | .85 | .75 | 3 |
| .0025 | .79 | .61 | 3 | .03 | .67 | .51 | 2 |
| | | | | .05 | .78 | .5 | 2 |

TABLE 2A

| | | | Patients Treated |
|---|---|---|---|
| | Normal | Untreated | with |
| | Subjects | Psychotic Patients | Antipsychotics |
| Significant UPOs | 67% | 60% | 50% |
| over surrogate | | | |
| files | | | |
| K values | 2.41 ± 0.46 | 2.56 ± 0.85 | 2.75 ± 0.32 |

These findings thus demonstrate the low-dimensional dynamics present in the ITIs of both normal and psychotic speech.

### EXAMPLE 2

### Analysis of Interword Time Intervals in Normal Subjects and Psychotic Patients using Fractal Methods

The determination of the fractal dimension of the chaotic process present in the ITIs directly from an experimental time series is used in order to gain information about the nature of the underlying dynamics. Comparison is then made between normal and psychotic subjects.

An additional quantitative method for analysis of the ITI vector is based on the calculation of the correlation dimension D_{c}. For this purpose, a set of n (n is also known as the “embedding dimension”) vectors Y₁, Y₂, . . . , Yₙ (representing an n-dimensional state space) is first constructed from the original data measurement vector. If the distance |Yᵢ−Yⱼ| (i,j=1, . . . , n) between two successive vectors Yᵢ, Yⱼ, is smaller than a predetermined value r, they are regarded as having a correlation. The correlation integral C(r,n) is expressed by:

$$C ( r , n ) = \frac{1}{{n}^{2}} \cdot {\sum }_{i = j}^{n} T ( r - \vert {Y}_{i} - {Y}_{j} \vert )$$

where T is a step function. The correlation dimension D_{c} of the set Y₁, Y₂, . . . , Yₙ, is given by:

$${D}_{c} = {\sum }_{r \to 0} \frac{ln [ C ( r , n ) ]}{ln ( r )} .$$

Therefore, the correlation dimension may be obtained by identifying a linear region on a ln[C(r,n)]-to-ln(r) plot curve.

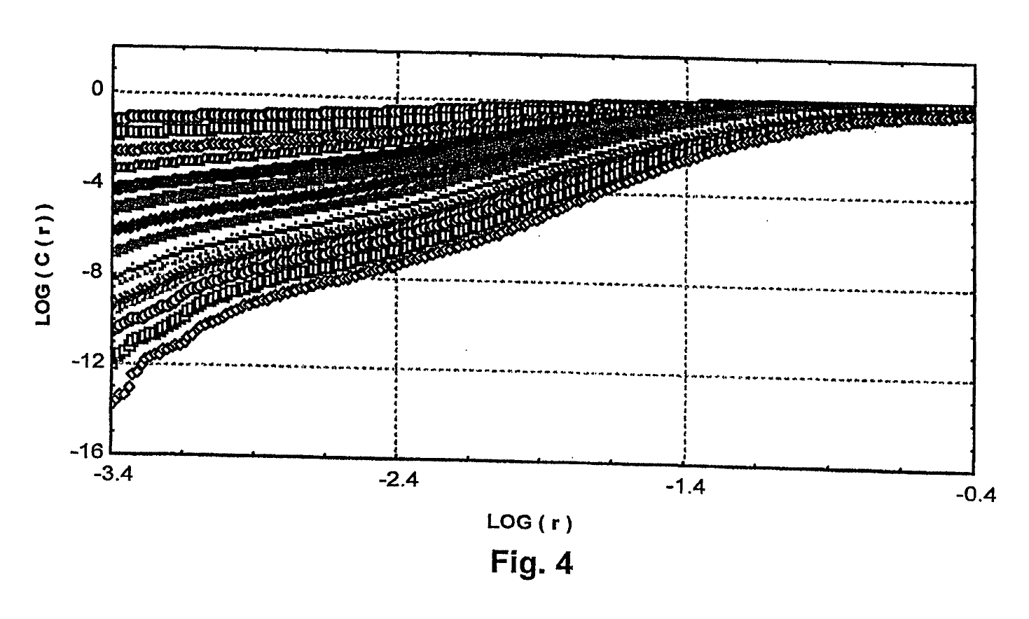

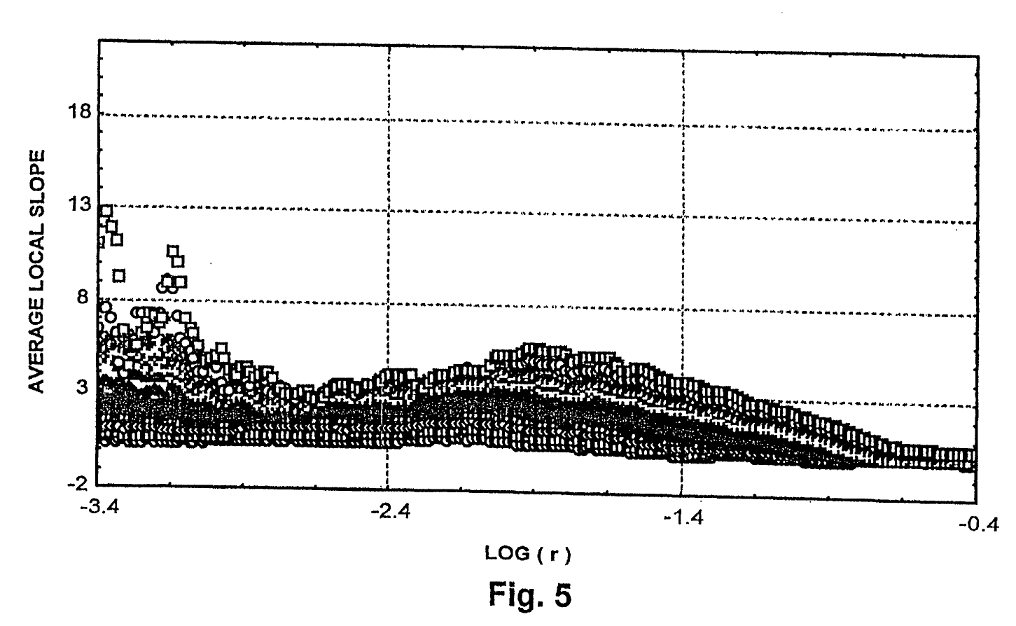

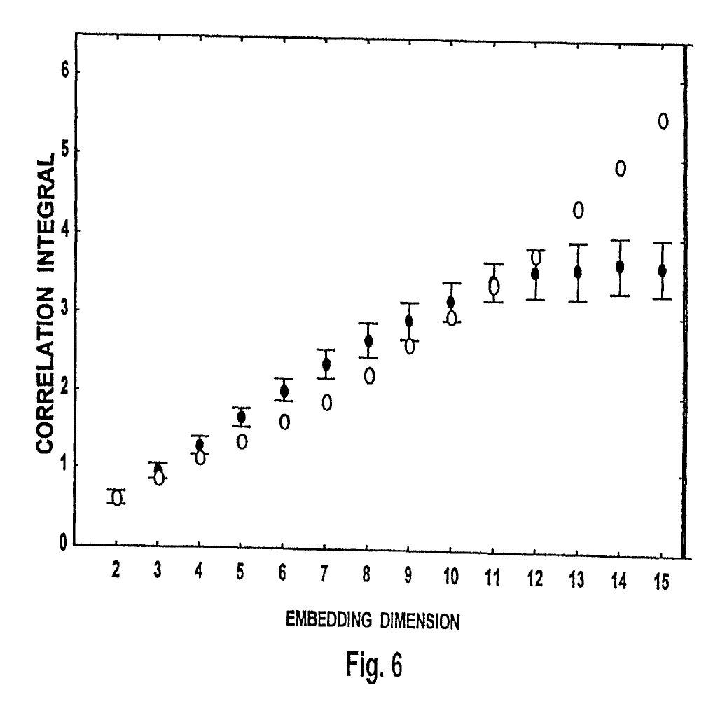

According to a preferred embodiment of the invention, the correlation dimension of the ITI vector is calculated by normalizing the ITI vector to a unity interval and reconstructing a two-dimensional state space (i.e. an embedding dimension of 2) using the normalized ITI vector. The correlation integral [C(r,n)] is then calculated for 300 different values of r for which 0.04<r<0.3, with a prefixed value of 0.015 for ln(rₘ/rₘ₊₁) where 1≦m≦299. FIG. 6 is a logarithmic plot of the correlation integral C(r,n) as a function of r for an embedding dimension value of two, with a linear region of essentially constant local slopes. FIG. 4 is a logarithmic plot of the correlation integral C(r,n) as a function of r for sever embedding dimension values ranging from 2-14, with a linear region of essentially constant local slopes. Next, the mean value of all local slopes within the linear region is calculated. FIG. 5 is a plot of the mean slope values as a function of ln(r) for several embedding dimension values ranging from 2-14.

This process is repeated several times, for higher values of embedding dimension n. As a result a set of correlation integrals (for each value of embedding dimension) together with a corresponding set of the mean values of all local slopes are obtained. Finally, the mean values of all local slopes are plotted as a function of the embedding dimension n. Inferences about the experimental data may be obtained from the convergence/divergence of these plots (normally, convergence and divergence represent chaotic signals and meaningless noise, respectively).

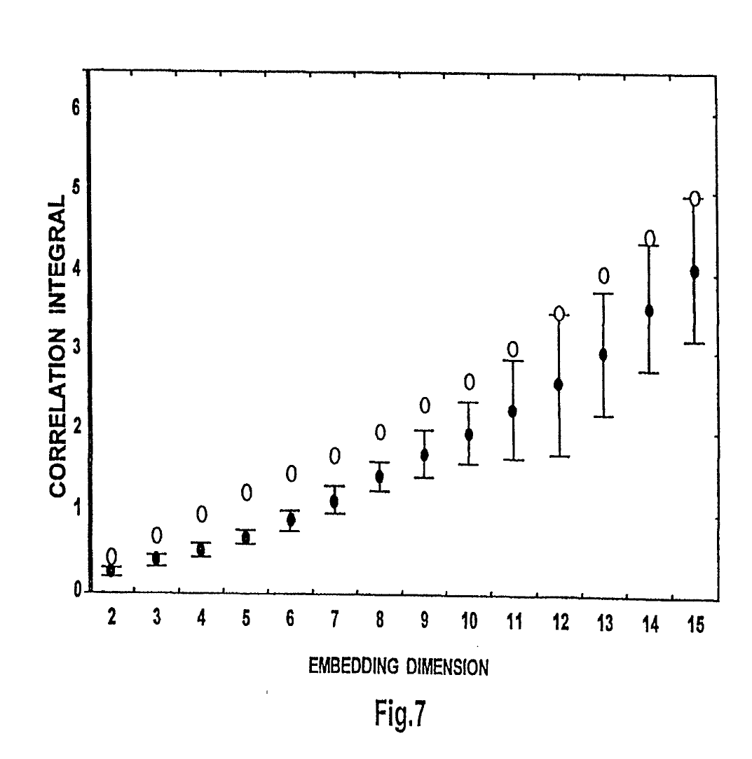

FIG. 6 is a representative plot of the correlation integral C(r,n) as a function of n, the embedding dimension for the ITIs of a normal subject (closed circles). With increasing n, D_{c} approaches a saturation value, Dₛ of about 3.5. A surrogate record of the same ITIs (open circles) resulted in a non-saturable curve, indicating a random time series. The corresponding plot for a psychotic patient is shown in FIG. 7. The correlation integral increases continually with increasing n, without reaching saturation (closed circles), indicating that the ITIs for the group of psychotic patient could not be described by low-dimensional chaotic attractors. A surrogate record (open circles) gives results very similar to the original plot. From these data, a clear difference between the normal subject and the psychotic patient is seen.

Relatively small data sets may be used in order to calculate the correlation dimension. The dimensionality of an attractor is a measure of the number of variables present in the evolution of a dynamic system (i.e. the number of degrees of freedom). Grassberger and Procaccia (Physical Review Letters 50: 346-349 (1983)) defined a distance distribution function, the correlation integral, C(r,n) in n-dimensional space, corresponding to the number of all distances between two points which are smaller than a given value of r. For small values of r, it is found that C(r,n) is proportional to r^{d}. If, with increasing values of n,d becomes independent of n (that is, it reaches a saturation value, dₛ), then the system under study can be said to possess a chaotic attractor with a dimension equal to dₛ. This dimension may then be used for the purposes of comparing the dynamics of two or more systems. In the present invention, these systems are the ITIs of psychotic patients and normal individuals.

In order to further investigate the differences between normal and psychotic individuals, the following groups were compared, with respect to parameters defining ITI non-linear dynamics:

a normal subjects;

b. psychotic patients;

c. treated psychotic patients.

The results are summarized in Table 2B.

TABLE 2B

| | | | Patients Treated |
|---|---|---|---|
| | Normal | Untreated | with |
| | Subjects | Psychotic Patients | Antipsychotics |
| Correlation | 87% | 7% | 100% |
| integral saturation | | | |
| Correlation | 3.2 ± 1.1 | — | 3.2 ± 1.3 |
| dimension | | | |

The results presented in Table 2B show that the normal subjects display characteristics of a low-dimensional chaotic attractor with dₛ=3.2. In contrast to the normal subjects, the group of psychotic patients is characterized by ITIs that could not be described by low-dimensional attractors. It is to be noted that unlike the dynamic behavior of the ITIs, the time vector of the words themselves was found to be random in both the control and psychotic subject groups. It is of interest that treated psychotic patients show saturation of the correlation integral, indicating reversion to the non-linear dynamics of non-psychotic individuals.

In summary, it appears that although normal speech is characterized by a low dimensional attractor, psychotic speech is found to be rather more complicated and less controlled, with a reconstruction of the speech attractor.

### EXAMPLE 3

### Analysis of Interword Time Intervals using Symbolic Dynamics

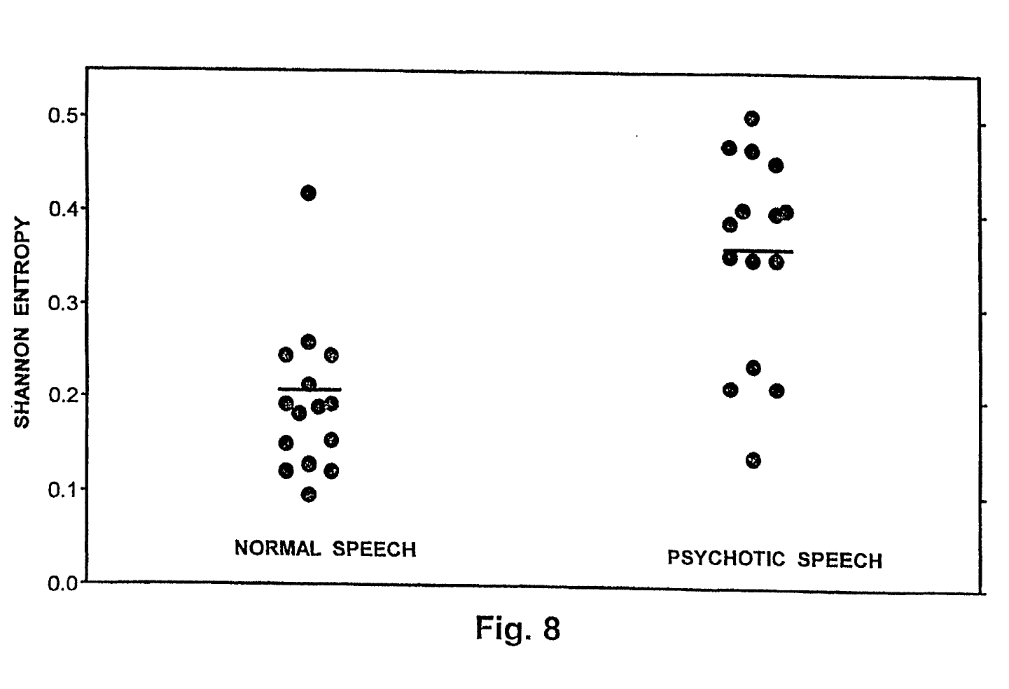

The concept of symbolic dynamics (Hao, B L, Physica D 51: 611-617 (1991)), is based on coarse-graining of the dynamics. The time series of ITIs are transformed into symbol sequences. For example, a set of predetermined ranges of measured values is defied and identified with a set of corresponding symbols. Any measured value that falls in a predetermined range is transformed to a symbol. The data is analyzed by seeking different symbol patterns. By comparing different kinds of such transformations, it was found that the use of four symbols was appropriate for the purposes of the present invention. Shannon Entropy is then calculated for groups of four symbols, so as to disclose particular frequencies of groups. The Shannon entropy calculated from the distribution of selective words is a suitable measure of the complexity of the time series. FIG. 8 shows that significantly higher values (and therefore higher complexities) were observed for psychotic (0.41±0.10) speech as compared with normal speech (0.22±0.05) (p<0.001, Mann-Whitney U-test). After calculating the Shannon Entropy for each individual, the Mann-Whitney U-test is used to find the difference between the group of normal subjects and the group of psychotic patients. The Shannon Entropy values are arranged according to an up-going series and inferences are obtained from the relative differences between the values, regardless their absolute values. Thus the calculation of Shannon Entropy through the use of symbolic dynamics, followed by a Mann-Whitney U-test, confirms that psychotic speech is indeed more complex than normal speech. The results are summarized in Table 2C.

TABLE 2C

| | | | Patients Treated |
|---|---|---|---|
| | Normal | Untreated | with |
| | Subjects | Psychotic Patients | Antipsychotics |
| Shannon Entropy | 0.22 ± 0.05 | 0.36 ± 0.11 | 0.34 ± 0.02 |

### EXAMPLE 4

### Experimental Results of Bi-Spectral Analysis of Interword Time Interval

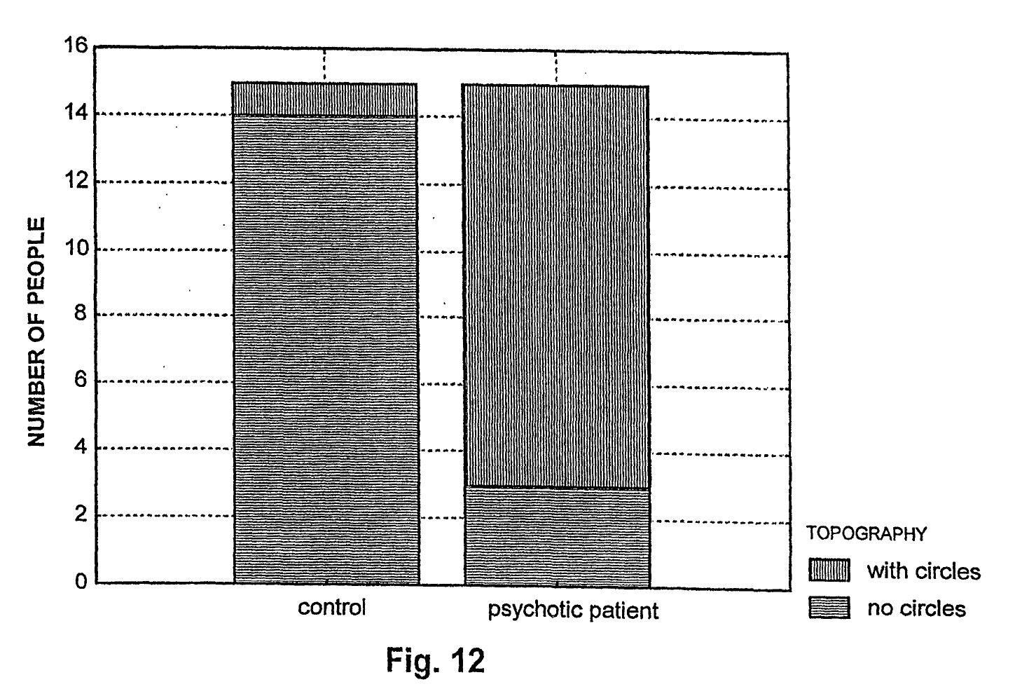

An experiment was carried out on a group of 15 psychiatric patients, and the topographic contours were extracted for each patient, using bi-spectral ITI analysis. FIG. 12 is a graph of the distribution of closed contour circle characteristics between 15 psychiatric patients and 15 normal (control) individuals, with a x² test which resulted in p=0.001. Hence, the probability that the difference between the results obtained for the group of psychotic patients and the group of normal subjects is not random is 0.999. A topography which is close to a normal type topography, was obtained among 3 from the 15 psychiatric patients. One psychiatric-type topography has been obtained among the group of 15 normal (control) subjects.

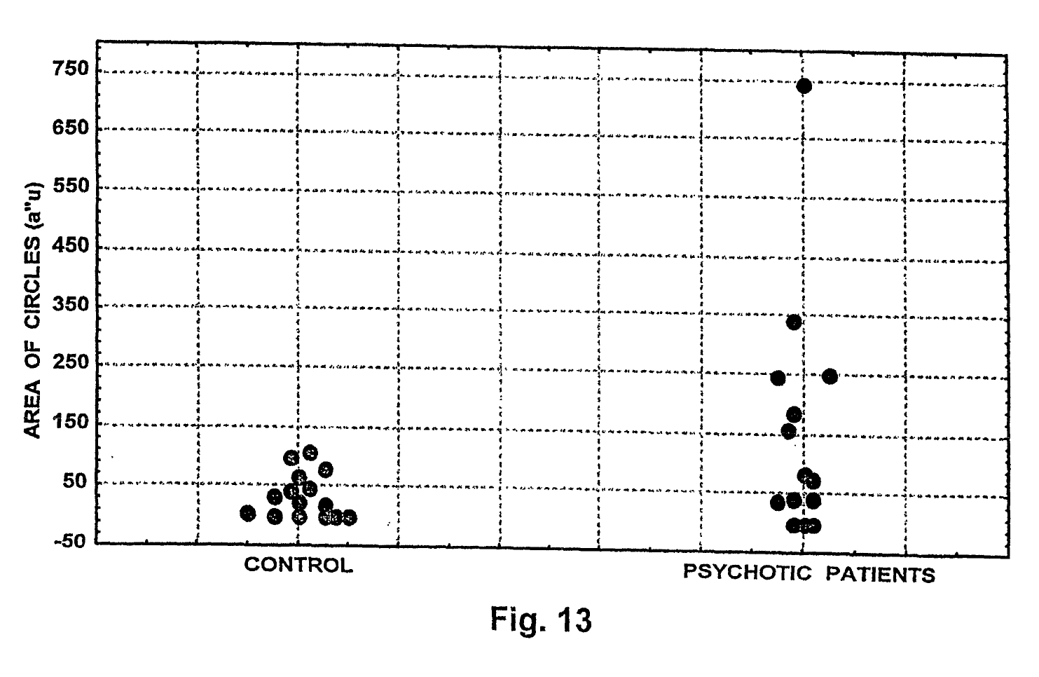

Another observation on the normalized bi-spectral analysis, which shows a great difference between normal and mentally ill subjects, is obtained by computing the number of points which are contained wit each closed contour. The result are shown as the area of circles within each contour, in FIG. 13. Statistical analysis of the results with the Mann-Whitney U test shows a significant difference between the circle areas found for each group of subjects (U=58.5, p=0.042). The results are summarized in Table 2D.

TABLE 2D

| | | | Patients Treated |
|---|---|---|---|
| | Normal | Untreated | with |
| | Subjects | Psychotic Patients | Antipsychotics |
| Bi-spectral | 6.7% | 80% | 0% |
| Analysis: Double | | | |
| circulatory forms | | | |

### EXAMPLE 5

### Interword Time Interval Analysis as a Tool for the Assessment of Child Development

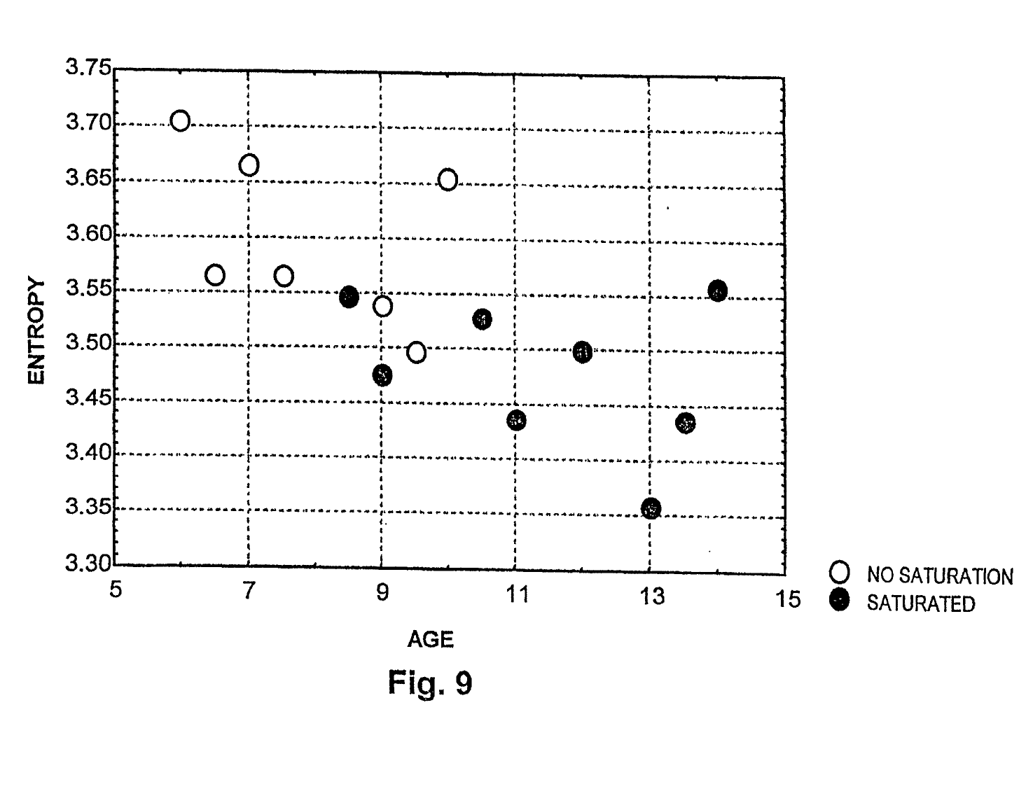

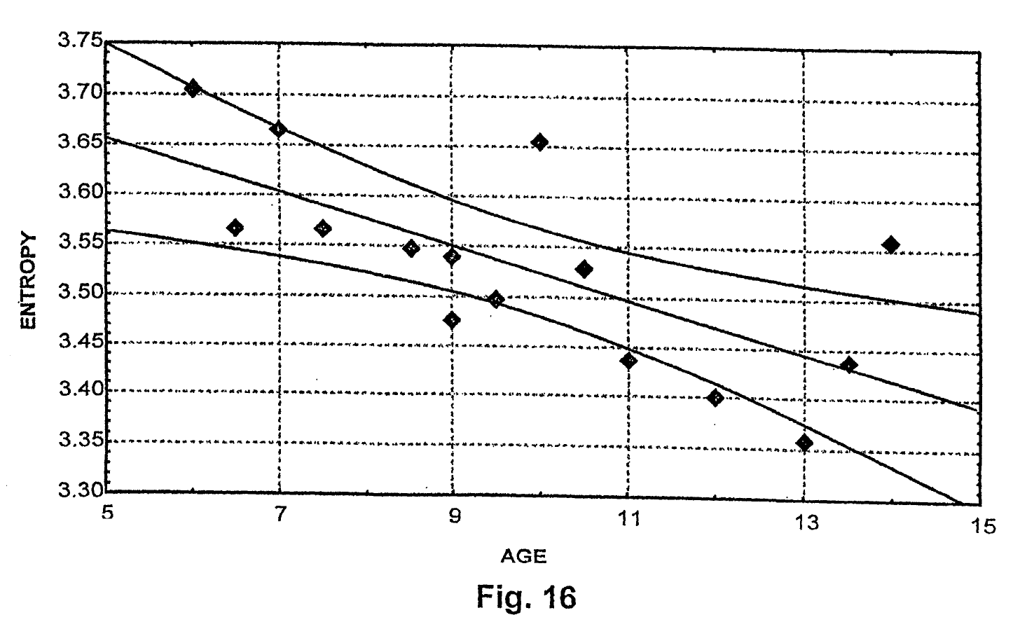

The speech of fifteen children aged 6 to 14 was recorded, and ITIs were analyzed by the same methods of correlation dimension analysis and symbolic dynamics entropy calculations as described in Examples 2 and 3 respectively. The results of these analyses are shown in FIG. 9. The correlation between symbolic dynamic entropy and age is was calculated as Pearson's coefficient of correlation (r=−0.6785; p=0.00547), which provides an indication about the degree of correlation between two variables by a linear approximation. High correlation is obtained if the correlation may be approximated by a straight line (also known as the regression line), with negative or positive slope. The regression curve for this relationship is shown in FIG. 16, showing 95% confidence limits. The results of both the entropy calculations and the correlation dimension analysis (i.e. presence or absence of saturaton) indicate that there is a trition from psychotic-type thought processes to normative adult-type thought processes at approximately age 8. The transition age obtained by ITI analysis is thus in agreement with that generally accepted in the field of developmental psychology. The analytical techniques of the present invention are therefore highly suitable for use in the assessment of child development and the early detection and diagnosis of learning and developmental problems.

### EXAMPLE 6

### Comparison between Different Analysis Methods

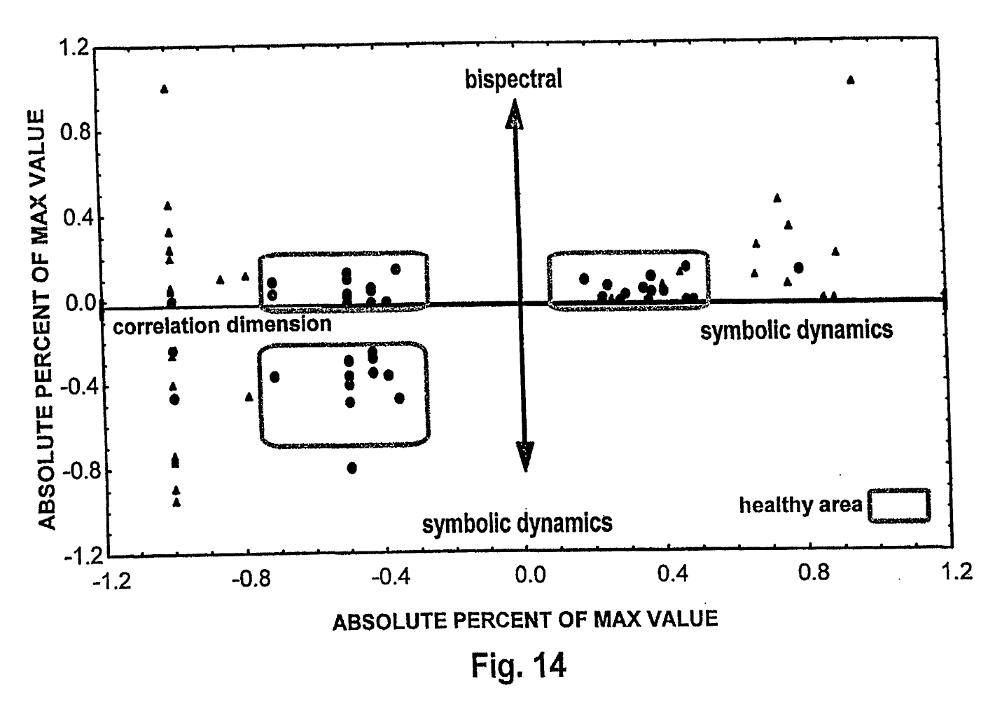

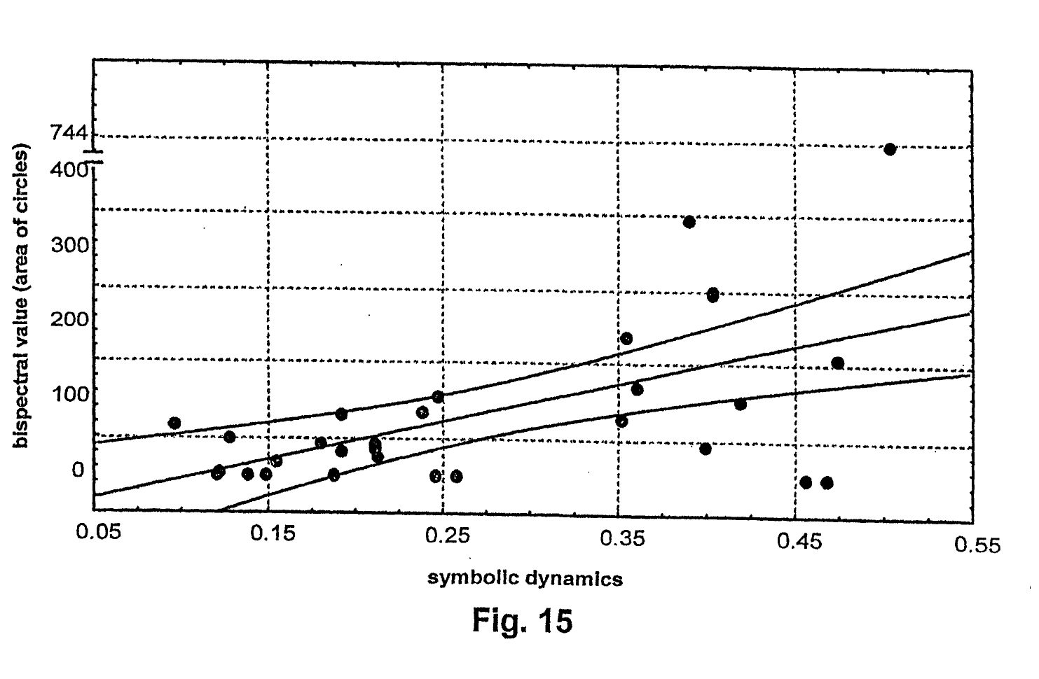

The results obtained from the application of the three methods described above (symbolic dynamics, correlation dimension and bi-spectral analysis) to the analysis of interword silences of normal individuals and psychiatric patients, were compared against each other pairwise. It may be seen from FIG. 14 that there is significant clustering of normal individuals in one region of the graph, and of psychiatric patients in a different region, when the results from pairs of the above tests are compared. The upper right frame of FIG. 14, showing the correlation of symbolic dynamics with bi-spectral analysis, is given in more detail in FIG. 15 (Pearson's r=0.55989; p=0.001621), showing regression with 95% confidence limits. The relationship between these two functions may be given as:

bi-spectral value=−89.08+671.96*symbolic dynamics.

### Experimental Apparatus

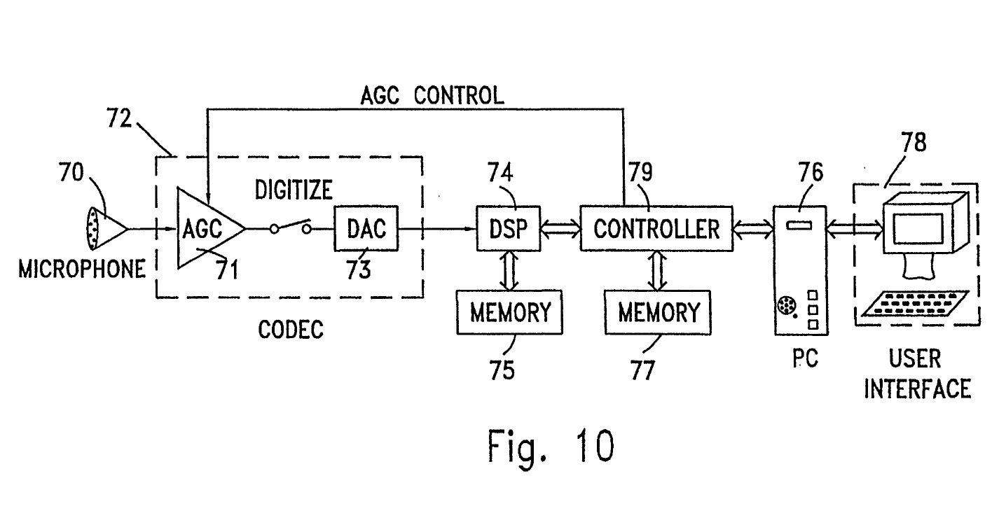

Examples 1 to 4 were carried out according to a preferred embodiment of the present invention, by using the experimental apparatus for the diagnosis of psychotic patients, by the analysis of Interword Time Intervals (ITIs), shown in FIG. 10. This experimental setup is described for the purpose of illustration only as the skilled person will be able to provide many different systems. Looking at the FIG. 10, the apparatus comprises a microphone 70, for converting the patient's voice to a series of electric signals and an automatic Gain Control (AGC) circuitry 71, for controlling the level of the electric signals transformed from the speech data. These electric signals am digitized by a Coder-Decoder (CODEC) 72, such as a suitable multi-media sound card, and then transformed into digital representation by an Analog to Digital Converter (A/D) 73. A digital signal processor (DSP) 74, is used for measuring the speech ITIs, analyzing the data and extracting the required parameters for further ITI analysis. The digital speech data, together with the processed data are stored in a memory 75, associated with the DSP 74. The parameters extracted by the DSP 74, are stored in a second memory. An analysis and computation unit, such as a Personal Computer (PC) 76 with a suitable software, uses the parameters stored in the second memory for patient characterization and interacts with the user by a user interface 78, such as a keyboard and a suitable display. In some cases, the Central Processing Unit (CPU) of the PC 76 provides the required digital signal processing instead of a DSP unit. An RS-232 data-bus 77 connects between the DSP 74 with its associated memory 75, the user interface 78, and the PC 76. The controller 79 associated with the second memory 77, which may be a micro-controller, controls the operations of the AGC, circuitry 71, the DSP 74, and its associated memory 75 and the data flowing in the connecting data-bus. Analysis results and/or the patient characterization parameters are displayed in conjunction with voice playback provided by a PC sound card.

According to a preferred embodiment of the invention, the rests obtained by using each of the analysis methods (UPOs, fractal methods, symbolic dynamics and bi-spectral analysis, described hereinabove) are combined into a generalized analysis method that enhances the sensitivity and the of each analysis method. For example, the analysis results related to a normal subject, may indicate that this subject is normal according to three (out of four) individual methods and that this subject is psychotic according to the fourth individual method. On the other hand, the analysis results related to another normal subject, may indicate that the latter subject is normal according to another three (out of four) individual methods and that this subject is psychotic according to the fourth individual method. The generalized analysis overcome these fluctuations by inferences which are based on the results of most individual analysis methods, while the opposite indication is considered to be a “measurement noise”. According to the generalized analysis, the results related to a normal subject indicate normal characteristics, even if the results of a single individual analysis indicate a psychotic state in the same normal subject.

Of course, the above examples and description has been provided only for the purpose of illustrations, and are not intended to limit the invention in any way. As will be appreciated by the skilled person, the invention can be carried out in a great variety of ways, employing more than one technique from those described above. Additionally, many different devices and apparatus can be provided for analyzing the ITIs, all without exceeding the scope of the invention.

1. A method for the analysis of thought states in a subject comprising measuring the interword time intervals (ITIs) of the subject and extracting parameters representative of the subject's ITI behavior from the measured ITI data.

2. A method according to claim 1, comprising:
a) recording the voice of the subject during his speech into a memory;
b) digitizing the recorded voice;
c) controlling the gain of the electrical signals representing the subject's voice;
d) transforming the digitized voice to a digital data representation;
e) measuring the interword time intervals (ITIs) from the digital data;
f) extracting parameters representative of the subject's ITI behavior from the measured ITI data; and
g) characterizing the subject by processing and analyzing his ITI data using the extracted parameters of step f) above.

3. A method according to claim 2, wherein the subject is characterized by calculating the correlation dimension based on his ITI data.

4. A method according to claim 3, comprising:
a) providing an original vector representing the ITI data measurement;
b) normalizing the values of said original vector to a unity time interval;
c) determining an embedding dimension;
d) generating a set of state vectors from said original vector, the dimension of state vectors in said set being equal to said embedding dimension;
e) determining a threshold distance between a pair of state vectors, below which said pair of state vectors being correlated;
f) calculating the correlation integral for said set of state vectors as a function of a predetermined range of threshold distances;
g) plotting the calculated values of said correlation integral as a function of said predetermined range of threshold distances, using a logarithmic-logarithmic scale;
h) identifying a linear region in said logarithmic-logarithmic plot and calculating the mean value of all local slopes within said identified linear region;
i) repeating steps c) to h) above for a plurality of different embedding dimensions being higher than said determined embedding dimension;
j) obtaining the correlation dimension function by plotting all mean values of all local slopes calculated for each embedding dimension as a function of the embedding dimension; and
k) characterizing the random or deterministic attributes of said original vector by identifying convergence or divergence of said correlation dimension function with a growing value of the embedding dimension.

5. A method according to any one of claims 1 to 4, wherein the thought state to be analyzed is a psychotic or psychotic-like disorder.

6. A method according to any one of claims 1 to 5, wherein psychotic patients are characterized by the divergence of their ITI data correlation dimension value.

7. A method according to claim 1, wherein the patient is characterized by a symbolic dynamics analysis of his ITI data.

8. A method according to claim 7, comprising:
a) providing an original vector representing the ITI data measurement;
b) dividing the range of said ITI data to a set of data intervals;
c) assigning a unique symbol to each data interval;
d) transforming the set data values from said original vector to a corresponding set of symbols by the ascription of said each data value to a corresponding interval from said set of data intervals, said data value being contained within said interval;
e) defining a group of symbols of fixed size from said set of symbols;
f) identifying the frequency of said defined group of symbols in said set of symbols by calculating the Shannon entropy for said group of symbols;
g) generating an upgoing series of said calculated Shannon entropy values; and
h) characterizing the random or deterministic attributes of said original vector by applying the Mann-Whitney test on said generated up-going series and finding differences between the group of normal subjects and the group of psychotic patients.

9. A method according to claim 7 or 8, wherein a patient is characterized as psychotic if the value of this Shannon entropy is greater than 0.3.

10. A method according to claim 1, wherein the patient state is characterized by finding and counting points of Unstable Periodic Orbits (UPOs) based on his ITI data.

11. A method according to claim 10, comprising:
a) generating an original vector from the ITI data measurement values;
b) constructing a three dimensional phase space containing a plurality of points in said space, said plurality of points being related to the values of said original vector;
c) determining the main diagonal in said phase space, said main diagonal representing the collection of all points in said phase having identical coordinates;
d) identifying and counting all points of UPOs in said original vector by seeking all sets of six consecutive points in said phase state, the corresponding distances of the first three points from said first set to said main diagonal defining a down-going series, the corresponding distances of the last three points from said first set to said main diagonal defining an up-going series;
e) generating a surrogate vector from said original vector by randomly scrambling the order of data values of said original vector;
f) identifying and counting all points of UPOs in said surrogate vector;
g) repeating steps e) and f) above a predetermined number of times;
h) calculating the mean value of all counts of points of UPOs over all generated surrogate vectors; and
i) characterizing the random or deterministic attributes of said original vector by comparing said mean value to the number of points of UPOs identified in said original vector.

12. A method according to claim 10, wherein the patient characterization is carried out by:
a) analyzing the motion of the state variables of a dynamical system representing the patient original ITI data;
b) measuring the number of encounters of this motion with UPOs;
c) generating a surrogate ITI data file constructed by using a random process;
d) measuring the number of encounters of the surrogate motion with UPOs;
e) defining a measure of significance, given by the absolute value of the difference between the number of original and surrogate encounters with UPOs, divided by the standard deviation of the surrogate values; and
f) calculating the error function of half the value of the measure of significance, representing the patient's p value.

13. A method according to claim 1, wherein the ITI data is used to analyze cognitive development stages in children.

14. A method according to claim 1, wherein the ITI data is used for the diagnosis of abnormal mental states.

15. A method according to claim 1, wherein the ITI data is used for the diagnosis of abnormal behavioral states.

16. A method according to claim 1, wherein the subject is characterized by performing bi-spectral analysis of his ITI data.

17. A method according to claim 16, wherein the bi-spectral characterization comprises:
a) computing the inter-modulation products of the sampled ITI data;
b) computing the triple product for each pair of Fourier frequency components of said inter-modulation products;
c) summing all the computed triple products of all pairs of Fourier frequency components;
d) obtaining the bi-spectrum by computing the magnitude of the sum of triple products;
e) computing the Real-Triple Product of all inter-modulation products;
f) normalizing said bi-spectrum to said Real-Triple Product;
g) generating a two-dimensional contour graph of said normalized bi-spectrum as a function of Fourier frequencies; and
h) obtaining the number of closed contours from said contour graph.

18. A method for the analysis of interword time intervals (ITIs) in a subject comprising:
a) recording the voice of the subject during his speech into a memory;
b) digitizing the recorded voice;
c) controlling the gain of the electrical signals representing the subject's voice;
d) transforming the digitized voice to a digital data representation;
e) measuring the interword time intervals (ITIs) from the digital data; and
f) extracting parameters representative of the subject's ITI behavior from the measured ITI data.

19. A method according to claim 1, comprising:
a) characterizing the subject by several different analysis methods of the measured ITI data, each of which provides an indication that corresponds to the thought state of said subject;
b) obtaining inferences related to thought states of said subject according to the indication which is common to most of said different analysis methods.

20. Apparatus for the diagnosis of psychotic patients by the analysis of Interword Time Intervals (ITIs) comprising:
a) a microphone for converting the patient's voice to a series of electric signals;
b) an Automatic Gain Control (AGC) circuitry for controlling the level of the electric signals representing the speech data;
c) a Coder-Decoder (CODEC) for digitizing the speech data;
d) a converter, for transforming the digitized speech data into digital representation;
e) a digital signal processor (DSP) for measuring the speech ITIs, analyzing the data and extracting the required parameters for further ITI analysis;
f) a memory associated with the digital signal processor, storing the digital speech data information together with the processed data;
g) a second memory, storing the parameters extracted according to step e) above;
h) analysis and computation unit for patient characterization based on the ITI data of step f) above and the stored parameters of step g) above;
i) user interface means for user interaction with analysis and computation unit;
j) communication means communicating between the digital processor with its associated memory, the user interface and the analysis and computation unit;
k) a controller associated with the second memory of step g) above, for controlling the operations of the AGC circuitry, the DSP and its associated memory and the communication means of step j) above;
l) display and/or printing means for displaying analysis results and/or the patient characterization parameters; and
m) optional voice playback means for representing the analysis and computation unit results.

21. Apparatus according to claim 20, wherein the analysis and computation unit comprises a Personal Computer (PC).

22. Apparatus according to claim 20, wherein the CODEC function is implemented using a suitable multi-media sound card.

23. Apparatus according to claim 20, wherein the PC Central Processing Unit (CPU) carries out the DSP and the controller operations.

24. Apparatus according to claim 20, wherein the controller is a micro-controller.

25. A method for the analysis of thought states in a subject, particularly for the determination of psychotic or psychotic-like disorders, essentially as described and illustrated.

26. Apparatus for the analysis of thought states in a subject, particularly for the determination of psychotic or psychotic-like disorders, essentially as described and illustrated.
